(19) United States
(12) Patent Application Publication
(10) Pub. No.: US 2020/0231924 A1
(43) Pub. Date: Jul. 23, 2020

(54) SUBSTRATE FOR TEST USE, AND METHOD FOR PRODUCING SUBSTRATE FOR TEST USE

(71) Applicants: IWASAKI ELECTRIC CO., LTD., Tokyo (JP); NATIONAL INSTITUTES FOR QUANTUM AND RADIOLOGICAL SCIENCE AND TECHNOLOGY, Chiba-shi (JP)

(72) Inventors: Shinobu KINOSHITA, Gyoda City (JP); Takashi IDE, Gyoda City (JP); Tomoko OYAMA, Takasaki-shi (JP); Mitsumasa TAGUCHI, Takasaki-shi (JP); Bin Jeremiah Duenas BARBA, Quezon City (PH)

(73) Assignees: IWASAKI ELECTRIC CO., LTD., Tokyo (JP); NATIONAL INSTITUTES FOR QUANTUM AND RADIOLOGICAL SCIENCE AND TECHNOLOGY, Chiba-shi (JP)

(21) Appl. No.: 16/620,400

(22) PCT Filed: May 17, 2018
(86) PCT No.: PCT/JP2018/019084

§ 371 Date: Dec. 6, 2019

(30) Foreign Application Priority Data
Jun. 8, 2017 (JP) 2017-113092

(51) Int. Cl. C12M 1/42; B01L 3/00; C08F 283/12; B01J 19/12; C12M 1/32

(52) U.S. Cl. CPC C12M 35/02; B01L 3/502707; B01L 3/5085; B01L 2300/163; B01J 19/12; C12M 23/12; B01L 2300/0864; C08F 283/124

(57) ABSTRACT

Provided are a substrate for test use that is preferable for use in a test such as a culture test, and a method for manufacturing the substrate for test use. The substrate for test use, in which a solution retaining part for retaining water or an aqueous solution, is formed at a surface of a substrate of polydimethylsiloxane (PDMS). The solution retaining part is a concave part having a hydrophilic surface layer. The surface layer has a maximum thickness of 1 μm or larger.

### CROSS-REFERENCE TO RELATED PATENT APPLICATIONS

This application is a U.S. National Phase Application under 35 U.S.C. § 371 of International Patent Application No. PCT/JP2018/019084, filed May 17, 2018, and claims the benefit of priority to Japanese Patent Application No. 2017-113092, filed on Jun. 8, 2017, all of which are incorporated herein by reference in their entirety. The International Application was published in Japanese on Dec. 13, 2018 as International Publication No. WO/2018/225473 under PCT Article 21(2).

### FIELD OF THE INVENTION

The present invention relates to a substrate for test use and a method for manufacturing a substrate for test use.

### BACKGROUND OF THE INVENTION

Examples of cell culturing instruments used for cell culturing and cell screening include various instruments such as dishes. Recently, polydimethylsiloxane (hereinafter referred to as “PDMS”) has been known as the substrate of a cell culturing instrument.

PDMS is a transparent material having no autofluorescence and inactive against cells, and thus preferably used as the substrate of a culturing instrument. In addition, PDMS is inexpensive and easy to fabricate, and is fabricated in shapes in accordance with purposes and used at experiment and research sites in medical and biological fields.

However, PDMS is hydrophobic and thus repels solution such as a culture media and cells, thereby encumbering cell adhesion. For this reason, when PDMS is used as a substrate, reforming processing is needed to provide hydrophilicity to the surface of the substrate.

Examples of known methods of performing the reforming processing include plasma surface processing and chemical processing using chemicals and the like (refer to Japanese Patent Laid-open No. 2011-111373 and Japanese Patent Laid-open No. 2006-181407, for example).

Another known technology performs electron beam irradiation processing on PDMS to improve the hydrophilicity of the surface of the substrate (refer to Dong-Woo Kang and seven others, “Electron Beam-Induced Modification of Poly (dimethyl siloxane)”, (South Korea), Polymer Korea, The Polymer Society of Korea, May 2011, Vol. 35, No. 2, p. 157-1601, for example).

### Technical Problem

The plasma surface processing is relatively easy to perform. However, hydrophobicity starts being restored right after the plasma surface processing, and hydrophilicity is lost in a relatively short time, which is a large defect. For this reason, the plasma surface processing needs to be performed right before cell culturing is started, and thus PDMS provided with the plasma surface processing cannot be shipped as a product nor stocked for a long period.

With chemical processing, hydrophilicity is maintained for a longer period than with the plasma surface processing, but the chemical processing is extremely complicate to perform and is not suitable for production, which is a problem. In addition, cell toxicity due to, for example, remaining used chemicals and elution thereof is concerned.

With electron beam irradiation, it is possible to reduce processing time and cost as compared to the chemical processing. However, when surface reforming is performed on a surface of a substrate as described in Dong-Woo Kang and seven others, “Electron Beam-Induced Modification of Poly (dimethyl siloxane)”, (South Korea), Polymer Korea, The Polymer Society of Korea, May 2011, Vol. 35, No. 2, p. 157-160, the substrate is hardened, loses elasticity, and becomes brittle or deforms, and thus is unsuitable as a substrate of a cell culturing instrument.

The present invention is intended to provide a substrate for test use that is preferable for use in a test such as a culture test, and a method for manufacturing the substrate for test use.

### SUMMARY OF THE INVENTION

### Solution to Problem

An aspect of the present invention is a substrate for test use in which a solution retaining part for retaining water or an aqueous solution is formed at a surface of a substrate of polydimethylsiloxane (PDMS), wherein the solution retaining part is a concave part having a hydrophilic surface layer, and the surface layer has a maximum thickness of 1 μm or larger.

According to another aspect of the present invention, in the above-described substrate for test use, the concave part has a maximum depth of 0.5 μm or larger.

According to another aspect of the present invention, in the above-described substrate for test use, the solution retaining part has wettability with a water contact angle of 90° or smaller.

Another aspect of the present invention is a method for manufacturing a substrate for test use, the method including an electron beam irradiation process of irradiating a substrate of polydimethylsiloxane (PDMS) with an electron beam to form a solution retaining part for retaining water or an aqueous solution, wherein, in the electron beam irradiation process, a place at which the solution retaining part is to be formed is irradiated with the electron beam at an acceleration voltage with which a concave part having a surface layer reformed to be hydrophilic is formed at a surface of the substrate.

According to another aspect of the present invention, in the above-described method for manufacturing a substrate for test use, the acceleration voltage is equal to or lower than 1 MV.

According to another aspect of the present invention, in the above-described method for manufacturing a substrate for test use, a radiation dose of the electron beam is equal to or larger than 2 MGy.

According to another aspect of the present invention, in the above-described method for manufacturing a substrate for test use, in the electron beam irradiation process, the surface of the substrate is irradiated with the electron beam in an atmosphere having an oxygen concentration equal to or higher than an oxygen concentration of air atmosphere.

### Advantageous Effects of Invention

The present disclosure indicates the following effects.

Specifically, in the present disclosure, a substrate for test use that is preferable for use in a test such as a culture test can be obtained because the substrate for test use is a substrate for test use in which a solution retaining part for retaining water or an aqueous solution is formed at the surface of a substrate of PDMS, the solution retaining part being a concave part having a hydrophilic surface layer, the surface layer having a maximum thickness of 1 μm or larger.

The present disclosure indicates that a practically usable substrate for test use including a solution retaining part can be obtained because the concave part has a maximum depth of 0.5 μm or larger.

The present disclosure indicates that a sufficiently hydrophilic solution retaining part can be obtained because the solution retaining part has wettability with a water contact angle of 90° or smaller.

The present disclosure indicates that a substrate for test use in which water or an aqueous solution can be solidly retained without being flowed out of a solution retaining part is obtained through electron beam irradiation, because, in an electron beam irradiation process of irradiating a substrate of PDMS with an electron beam to form a solution retaining part for retaining water or an aqueous solution, a place at which the solution retaining part is to be formed is irradiated with the electron beam at an acceleration voltage with which a concave part having a surface layer reformed to be hydrophilic is formed at the surface of the substrate.

The present disclosure indicates that, when the acceleration voltage is set to be 1 MV or lower, the substrate is not hardened and does not lose elasticity nor suffer yellowing, curvature, and cracking, and thus workability thereof can be excellently maintained.

The present disclosure indicates that, when the radiation dose of the electron beam is set to be 2 MGy or larger, the water contact angle can be 90° or smaller.

The present disclosure indicates that oxidation at an electron beam irradiation place can be promoted by irradiating the surface of the substrate with the electron beam at an oxygen concentration equal to or higher than that of air atmosphere, thereby efficiently achieving high hydrophilicity.

### BRIEF DESCRIPTION OF THE DRAWINGS

FIGS. 1A and 1B are diagrams illustrating a configuration of a cell culturing instrument according to an embodiment of the present invention: FIG. 1A is an overall view and FIG. 1B is an enlarged view of Part A in FIG. 1A.

FIG. 2 is a pattern diagram illustrating a well section configuration together with a culture media.

FIG. 3 is a graph illustrating the relation between the radiation dose of an electron beam and the water contact angle.

FIG. 4 is a graph illustrating results of measurement of temporal change of the water contact angle.

FIG. 5 is a table indicating the relation among acceleration voltage, the radiation dose, and the water contact angle.

FIG. 6 is a diagram illustrating a configuration of a micro flow path chip according to a modification of the present invention.

### DETAILED DESCRIPTION OF THE INVENTION

The following describes an embodiment of the present invention with reference to the accompanying drawings. In the present embodiment, a cell culturing instrument used for cell culturing is described as an exemplary substrate for test use.

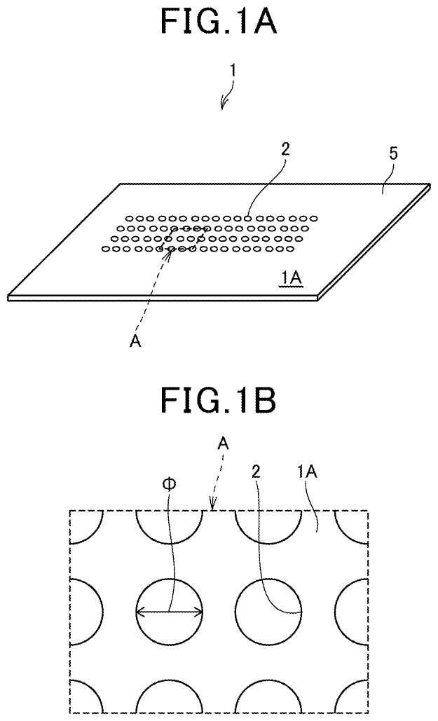

FIGS. 1A and 1B are diagrams illustrating a configuration of a cell culturing instrument 1 according to the present embodiment: FIG. 1A is an overall view and FIG. 1B is an enlarged view of Part A in FIG. 1A.

As illustrated in FIGS. 1A and 1B, in the cell culturing instrument 1, a well formation surface 1A that is a surface of a substrate 5 is provided with a plurality of wells 2 that are concave relative to the well formation surface 1A. In other words, the well formation surface 1A of the substrate 5 includes the wells 2 and a non-well part.

The shape of the cell culturing instrument 1 is not particularly limited, but the well formation surface 1A is preferably planer with taken into consideration the retainability of a culture media such as water or an aqueous solution in the wells 2. Examples of the shape of the cell culturing instrument 1 include a sheet shape, a plate shape, and a flat-bottomed dish shape. Specific examples of the cell culturing instrument 1 having a plate shape include a prepared slide and a cover glass, and specific examples of the cell culturing instrument 1 having a flat-bottomed dish shape include a microtiter plate.

In the present embodiment, the average thickness of the substrate 5, in other words, the distance from the well formation surface 1A to a surface (back surface) 1B on the opposite side is 50 μm or larger (preferably, 1 mm or larger) approximately, but may be any other thickness.

The substrate 5 of the cell culturing instrument 1 is formed of polydimethylsiloxane (PDMS). The substrate 5 may contain another material as long as PDMS is the primary component. The “primary component” means that an amount of PDMS contained in the substrate 5 is equal to or larger than 50 mass %.

In the present embodiment, the amount of PDMS contained in the substrate 5 is preferably as large as possible, and is preferably 75 mass % or larger, 90 mass % or larger, or 99 mass % or larger.

In addition, the substrate 5 may contain a conventionally well-known additive (for example, plasticizer) as a material other than PDMS as appropriate.

The substrate 5 containing PDMS as the primary component typically has a water contact angle of 100° approximately. In the present specification, the water contact angle is what is called a parameter indicating the degree of wettability and can be measured by a static droplet method.

A value and a material that are preferable in accordance with usage of the cell culturing instrument 1 and the like are selected as the amount of contained PDMS and the additive, respectively, as appropriate.

The shape of each well 2 formed in the above-described cell culturing instrument 1 only needs to be concave relative to the well formation surface 1A, and other features of the shape of the well 2 are optional.

For example, in the present embodiment, as illustrated in FIG. 1A, the well 2 has a circular opening shape in a plan view of the well formation surface 1A of the cell culturing instrument 1, but may have an optional shape such as an elliptical shape, a rectangular shape, a polygonal shape, a line shape, or a shape obtained by combining these shapes.

In the present embodiment, the shape of the bottom surface of the well 2 has a U-shaped section but may have, for example, a V-shaped section or a flat shape. It is relatively easy to manufacture the well 2 having a bottom surface in a U-shaped section.

In the present embodiment, each well 2 is formed in a size having an opening diameter ϕ (diameter) of 5 μm or larger and a depth d of 0.5 μm or larger at maximum (hereinafter simply referred to as “maximum depth”). The size of the well 2 is not limited thereto, but may be set as appropriate in accordance with usage. For example, when the opening shape is circular, the opening diameter ϕ being at least 5 μm or larger is sufficient for practical use and may be set to be 10 μm or larger or 100 μm or larger. The maximum depth of the well 2 being at least 0.5 μm or larger is sufficient for practical use irrespective of the opening shape and the sectional shape and may be set to be 1 μm or larger or 5 μm or larger. The upper limit of the maximum depth of the well 2 is not particularly limited but may be the smaller one of half the average thickness of the substrate 5 or smaller and 100 μm or smaller.

As described above, parameters such as the opening shape, bottom surface shape, opening size, and maximum depth of the well 2 may be set in an appropriate combination in accordance with usage of the cell culturing instrument 1.

For example, liquid (culture media) in amount in accordance with a purpose can be retained in the well 2 by adjusting the parameters as appropriate.

For example, when the cell culturing instrument 1 is used for single-cell culturing (culturing with single cell per well), the cell culturing instrument 1 including the well 2 in a circular shape in which the opening diameter ϕ is 5 μm to 100 μm approximately and the maximum depth is 1 μm to 50 μm approximately can be preferably used.

As illustrated in FIG. 1A, the multiple wells 2 are formed on the surface of the cell culturing instrument 1 according to the present embodiment. The number of wells 2 is, for example, 12 or larger, preferably 90 or larger, more preferably 300 or larger, still more preferably 400 or larger. The number of wells 2 is not limited thereto but only needs to be at least one.

In the present embodiment, the wells 2 are disposed in a lattice shape as illustrated in FIG. 1A. However, the disposition does not need to be in a lattice shape but is possible in an aspect in which the distance between the centers (barycenters) of adjacent wells is substantially constant. With such a disposition aspect, a larger number of wells 2 can be disposed at the well formation surface 1A of the substrate 5 of the cell culturing instrument 1.

The disposition aspect of the well 2 is not limited thereto but may be optionally set in accordance with usage of the cell culturing instrument 1 or the like.

Examples of a preferable aspect of the above-described cell culturing instrument 1 include an aspect in which about 400 wells 2 each in a circular shape having an opening diameter of 300 μm or larger (for example, 350 μm) are disposed in a circle having a diameter of 7 mm.

The bottom surface of a typically available 96-well microtiter plate (flat bottom) has a diameter of 7 mm approximately, and thus, according to the above-described aspect, it is possible to achieve disposition of about 400 wells 2 for each well of the microtiter plate. Thus, the cell culturing instrument 1 including about 40000 wells 2 in total can be easily produced. Such a cell culturing instrument 1, to which a typically available general-purpose experiment instrument is easily applicable, is excellent in handling.

Each well 2 formed in the cell culturing instrument 1 according to the present embodiment includes a hydrophilic surface layer 6 at the outermost surface (surface that contacts liquid injected into the well 2) of the well 2. In the present specification, “hydrophilic” means that the water contact angle is smaller than at a place on which hydrophilic processing is not performed (that is, a non-well part of the well formation surface 1A). Thus, when the surface layer 6 of the well 2 of the cell culturing instrument 1 is hydrophilic, the surface layer 6 has a water contact angle smaller than that of a part other than the surface layer 6 (at least a part other than the well 2).

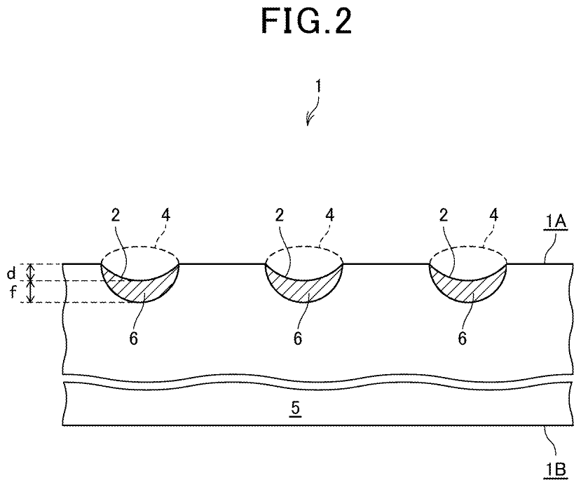

FIG. 2 is a pattern diagram illustrating a section configuration of each well 2 together with a culture media 4. The well 2 functions as a solution retaining part for retaining liquid (typically, culture media) in a concave part thereof. Since the well 2 includes the hydrophilic surface layer 6, the culture media 4 can be solidly retained (trapped) in the individual well. In addition, the difference in wettability between a substrate part other than the wells 2 and each well 2 (surface layer 6) can be exploited to use the substrate part other than the well 2 as a barrier for retaining the culture media only in the wells 2, in other words, for preventing the culture media (cell) from moving between the wells 2. Accordingly, when the cell culturing instrument 1 is immersed in liquid, droplets can be easily adhered to all wells 2, thereby easily obtaining what is called a droplet array.

The water contact angle of the surface layer 6 is preferably smaller than 90°, more preferably smaller than 80°. However, it is known that a too small water contact angle adversely affects the cell adhesion property. Thus, the water contact angle of the surface layer 6 of the well 2 is preferably equal to or larger than 20°, more preferably equal to or larger than 40°.

In the cell culturing instrument 1 according to the present embodiment, the hydrophilicity of the solution retaining part (which is the well 2) is maintained for a long time as compared to PDMS provided with hydrophilic processing through typical plasma irradiation. Specifically, the hydrophilicity with a water contact angle (for example, 80° or smaller) equivalent to that after hydrophilic processing (electron beam irradiation) is maintained for at least 3 days in a culturing environment (condition with immersion in the culture media at 37° C.). In the present specification, “after hydrophilic processing” means “within one hour after electron beam irradiation”, and “equivalent” means “error in 15% or smaller is allowed”.

Typically, the hydrophilicity retaining duration is preferable as long as possible and is, for example, 5 days or longer, 10 days or longer, 20 days or longer, 30 days or longer, or 50 days or longer in the present embodiment.

Specifically, the inventors have found that the duration in which the hydrophilicity (wettability) of the surface layer 6 is maintained can be increased by increasing in the thickness of the surface layer 6. A maximum thickness f of the thickness of the surface layer 6 being at least 1 μm or larger is sufficient for practical use, and is more preferably 10 μm or larger.

The upper limit of the maximum thickness f of the surface layer 6 is not particularly limited, but when the surface layer 6 is too thick, the substrate 5 potentially suffers hardening, elasticity decrease, yellowing, curvature, cracking, and the like. For example, the maximum thickness f of the surface layer 6 is preferably the smaller one of half the thickness from the deepest part of each recess to the back surface 1B of the substrate or smaller and 100 μm.

The thickness of the surface layer 6 can be measured by performing chemical composition analysis of a section thereof by microscope FT-IR, XPS, or the like.

In the cell culturing instrument 1, the above-described hydrophobic PDMS is used for the substrate 5. A method for manufacturing the cell culturing instrument 1 includes an electron beam irradiation process of irradiating the well formation surface 1A of the substrate 5 with an electron beam by an electron beam irradiation device, and the wells 2 are formed through the electron beam irradiation process.

Specifically, when the well formation surface 1A of the substrate 5 of PDMS is irradiated with the electron beam in an oxygen containing atmosphere, oxidation occurs at electron beam irradiation places, a methyl group contained in PDMS is discharged and bonded with an oxygen atom. Accordingly, various polar groups are held at the electron beam irradiation places, and the electron beam irradiation places are reformed to be hydrophilic.

In addition, the electron beam irradiation places contract because of, for example, the electron beam, crosslinking and decomposition due to heat generated through the irradiation, and gas discharge and molecule rearrangement along therewith, and accordingly, the electron beam irradiation places deform into concave parts.

Thus, in the electron beam irradiation process, a place where each well 2 is to be formed is irradiated with the electron beam to obtain the concave part shaped well 2 including the hydrophilic surface layer 6.

The irradiation of the substrate 5 of PDMS with the electron beam in the electron beam irradiation process is not limited to a particular aspect as long as the formation place of each well 2 is irradiated with the electron beam, but for example, an aspect in which electron beam irradiation is performed while a mask having a pattern and a shape in accordance with the shape and size of the well 2 is disposed on the surface of the substrate 5 may be applied.

Typically known schemes of the electron beam irradiation include a fixed irradiation scheme in which the electron beam irradiation place is not moved on the well formation surface 1A of the substrate 5 and a scanning scheme in which the electron beam irradiation place is moved to scan the well formation surface 1A. In a manufacturing process according to the present embodiment, any of the scanning scheme and the fixed irradiation scheme can be employed. In the electron beam irradiation, the above-described oxidation at the electron beam irradiation place can be promoted by irradiating PDMS at an oxygen concentration equal to or higher than that of air atmosphere (in other words, in an oxygen-rich atmosphere), thereby efficiently achieving high hydrophilicity. For example, oxygen may be supplied to the electron beam irradiation place by blowing the oxygen or the like so that the electron beam irradiation place is maintained in an oxygen-rich atmosphere. The oxygen concentration is preferably 50% or higher, more preferably 95% or higher, but may be set to another value depending on various conditions and the like.

The inventors have surprisingly found that the thickness of the surface layer 6 is proportional to electron beam acceleration voltage.

Specifically, as the acceleration voltage is higher, the electron beam reaches a deeper range through the well formation surface 1A, and hydrophilic reforming is achieved up to the range. Although described later in detail, the inventors have found that the hydrophilicity is maintained for a longer duration as the surface layer 6 is thicker, and for example, the hydrophilicity is maintained for at least 50 days or longer in a cell culturing environment when the maximum thickness f of the hydrophilic surface layer 6 is 40 μm approximately.

In culturing of a living body tissue used in regeneration medicine or the like, the culturing duration of two weeks to one month approximately is expected. When the maximum thickness f of the hydrophilic surface layer 6 is set to be 20 μm approximately, the cell culturing instrument 1 capable of stably performing culturing for an extremely long duration can be obtained. However, when the electron beam acceleration voltage is too high, the electron beam transmits to the back surface 1B (FIG. 2) of the substrate 5, which potentially causes hardening, yellowing, curvature, cracking, and the like of the substrate 5.

The electron beam acceleration voltage is preferably 1 MV or lower, more preferably 0.5 MV or lower, but may be set to be a value out of the ranges depending on various conditions. With such an acceleration voltage, the surface layer 6 having a preferable maximum thickness f (for example, 1 μm to 100 μm inclusive) can be relatively easily formed. The lower limit of the electron beam acceleration voltage is preferably 0.03 MV or higher but may be set to be a value out of the range depending on various conditions.

In addition, the inventors have surprisingly found that the maximum depth of each well 2 increases as the radiation dose of electron beam irradiation increases, but the water contact angle decreases as the radiation dose of electron beam irradiation increases. Thus, the maximum depth and hydrophilicity (water contact angle) of the well 2 can be adjusted to desired values by setting the radiation dose of electron beam irradiation as appropriate. The radiation dose of the electron beam is preferably 2 MGy or larger, more preferably 4 MGy or larger, but may be set to be a radiation dose out of the ranges depending on various conditions. With the radiation dose of the electron beam in the ranges, a cell culturing instrument having a preferable maximum depth of the well and a preferable water contact angle can be relatively easily formed. The upper limit of the radiation dose of the electron beam is preferably 100 MGy or smaller, but may be set to be a radiation dose out of the range depending on various conditions.

According to the above description, a preferable irradiation condition in the electron beam irradiation process is a condition that the acceleration voltage is equal to or lower than 1 MV and the radiation dose of the electron beam is 2 MGy or larger, more preferably a condition that the acceleration voltage is equal to or lower than 0.5 MV and the radiation dose of the electron beam is 4 MGy or larger.

With such an irradiation condition, the cell culturing instrument 1 in which the above-described wells 2 are formed can be obtained without losing the elasticity and quality of the substrate 5. Accordingly, the water contact angle, long-period maintenance of the hydrophilicity, and the maximum depth of the well 2 can be achieved at a preferable balance.

The acceleration voltage and the radiation dose of the electron beam under the above-described preferable irradiation condition may be set to be other values depending on various conditions.

In the manufacturing method according to the present embodiment, since the formation of each well 2 in a concave shape at the surface of the substrate 5 and the hydrophilic processing of the surface layer 6 of the well 2 simultaneously proceed, an easier manufacturing process is achieved than in a conventional manufacturing method in which these processes are performed at separate steps.

In the electron beam irradiation process, an ultra-micro well (having a diameter of 30 μm or smaller, for example) that is the well 2 having an extremely small size can be formed by controlling the spot diameter of the electron beam irradiation place or the like.

With a conventional method (for example, a combination of imprint and plasma irradiation, or chemical processing), misalignment is likely to occur between the concave part of the well 2 and a place reformed to be hydrophilic through hydrophilic processing, and thus it is difficult to form the well 2 having an ultra-micro size in which the surface layer 6 is reformed to be hydrophilic.

When the well 2 is formed by forming a concave part at the surface of a substrate through imprint and irradiating the concave part with plasma to achieve hydrophilic processing, it is difficult to deeply form the concave part through imprint, and the plasma is unlikely to reach a deep part of the concave part even when the concave part is deeply formed, and thus it is difficult to control the depth of the well 2 and the thickness of the surface layer 6 reformed to be hydrophilic to desired ranges.

However, with the manufacturing method according to the present embodiment, it is possible to control the depth of the well 2 and the thickness of the surface layer 6 reformed to be hydrophilic to desired ranges by controlling the acceleration voltage and/or the radiation dose of the electron beam in the electron beam irradiation process as described above.

Through such control, for example, the duration of factory production, shipment, and distribution of the cell culturing instrument 1 can be taken into consideration to maintain the hydrophilicity of the surface layer 6 of the well 2 for a long period exceeding at least the duration.

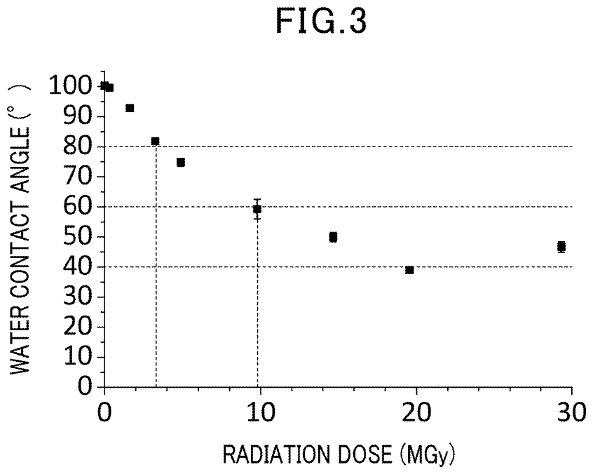

FIG. 3 is a graph illustrating the relation between the radiation dose of the electron beam and the water contact angle.

The graph of FIG. 3 was obtained through experiment, and in the experiment, PDMS fabricated in a sheet shape having a thickness of 1 mm to 2 mm approximately was used as a specimen. The specimen was irradiated with electron beam at the acceleration voltage of 55 kV by using the electron beam irradiation device of the fixed irradiation format that is capable of radiating an electron beam at the acceleration voltage of several hundred kV approximately.

As illustrated in FIG. 3, the water contact angle of the specimen was 100° approximately while no electron beam irradiation was performed (radiation dose=“0”). The water contact angle tended to decrease in proportional to the radiation dose, and the decrease of the water contact angle tended to stop in the range of 40° to 60° in the range of 10 MGy approximately or larger.

Thus, when the well 2 is formed through electron beam irradiation, it can be seen that the hydrophilicity of the surface layer 6 of the well 2 can be maximized by setting the radiation dose of the electron beam to be 10 MGy or larger.

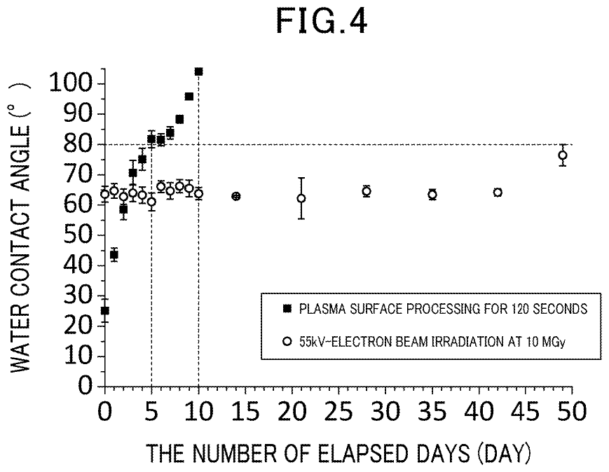

FIG. 4 is a diagram illustrating results of measurement of temporal change of the water contact angle.

In this measurement, the water contact angle of the specimen produced with the radiation dose set to be 10 MGy in the experiment of FIG. 3 was measured for 50 days. For measurement in a typical cell culturing environment, the specimen was immersed in a Dulbecco's modified Eagle media (DMEM) and stored for a certain duration while the temperature was maintained at 37° C., and then the measurement of the water contact angle was performed. In addition, temporal change of the water contact angle was also measured for a specimen produced through plasma surface processing (processing time: 120 seconds).

As illustrated in FIG. 4, the water contact angle of the specimen produced through the plasma surface processing rapidly increases (the hydrophilicity decreases) with time elapse, the water contact angle exceeds 80° at elapse of 5 days and reaches a value equivalent to that of PDMS irradiated with no electron beam at elapse of 10 days, which indicates loss of the hydrophilicity.

However, in the specimen produced through the electron beam irradiation, the water contact angle is kept at 80° approximately or smaller at elapse of 50 days, which indicates maintenance of the hydrophilicity of the well 2 for an extremely long duration.

The maximum thickness f of the surface layer 6 of the well 2 was equal to or smaller than several hundred nm in the specimen produced through the plasma surface processing, but the maximum thickness f was 40 μm approximately in the specimen produced through the electron beam irradiation at 55 kV. It is thought that such difference in the maximum thickness f of the surface layer 6 largely contributes to the hydrophilicity maintenance time.

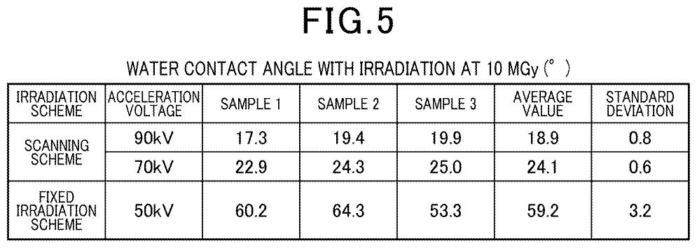

FIG. 5 is a table indicating the relation between the acceleration voltage and the water contact angle.

The inventors produced three specimens of Samples 1, 2, and 3 for each acceleration voltage of 90 kV or 70 kV by irradiating PDMS same as that in the experiment illustrated in FIG. 3 with the electron beam by the scanning scheme by using the electron beam irradiation device. The electron beam irradiation by the scanning scheme was repeated 10 times in total for the electron beam irradiation time of 30 seconds approximately at each repetition.

In addition, for comparison, three specimens of Samples 1, 2, and 3 were produced at the acceleration voltage of 50 kV by using the electron beam irradiation device of the fixed irradiation scheme, which is same as that in the experiment illustrated in FIG. 3.

The radiation dose is 10 MGy for each sample.

Results of measurement of the water contact angle after a certain duration passed since the production are listed in FIG. 5.

As illustrated in FIG. 5, the water contact angle is largely smaller than 90° at the acceleration voltage of 50 kV to 90 kV, which indicates that sufficient hydrophilicity is obtained.

The present embodiment achieves effects as follows.

In the present embodiment, in the electron beam irradiation process of irradiating the substrate 5 of PDMS with the electron beam to form the solution retaining part for retaining water or an aqueous solution (the well 2), the electron beam irradiation is performed at an acceleration voltage with which the concave part having the surface layer 6 reformed to be hydrophilic is formed.

Accordingly, the cell culturing instrument 1 in which water or an aqueous solution can be solidly retained without being flowed out of the solution retaining part can be obtained through the electron beam irradiation.

In addition, in the present embodiment, since the acceleration voltage is restricted to the relatively low energy of 1 MV or lower, the substrate 5 of PDMS is not hardened and does not lose elasticity nor suffer yellowing, curvature, and cracking, and thus workability thereof can be excellently maintained.

In addition, in the present embodiment, since the radiation dose of the electron beam is 2 MGy or larger, the solution retaining part sufficiently reformed to be hydrophilic can be formed when the well 2 is formed through electron beam irradiation.

In addition, in the present embodiment, since the well formation surface 1A of the substrate 5 of PDMS is irradiated with the electron beam in an atmosphere having an oxygen concentration equal to or higher than an oxygen concentration of air atmosphere, oxidation at the electron beam irradiation place is promoted, thereby efficiently achieving high hydrophilicity.

In addition, in the present embodiment, since the maximum thickness f of the surface layer 6 of the well 2 is equal to or larger than 1 μm, the cell culturing instrument 1 including the practically usable well 2 is achieved. Thus, the cell culturing instrument 1 in which the hydrophilicity is maintained for a long period is obtained. Such a cell culturing instrument 1 is preferable for culturing for a long period.

In addition, in the present embodiment, since the concave part of the well 2 has a maximum depth of 0.5 μm or larger, the cell culturing instrument 1 including the practically usable well 2 is achieved.

In addition, in the present embodiment, since the well 2 formed at the substrate 5 of PDMS has wettability with a water contact angle of 90° or smaller, the cell culturing instrument 1 in which the sufficiently hydrophilic well 2 is locally formed at the well formation surface 1A is obtained.

The above-described embodiment is merely an exemplary aspect of the present invention and may be optionally deformed and modified without departing from the scope of the present invention.

In the above-described embodiment, the shape of the well 2 is circular in plan view, but the present invention is not limited thereto, and the shape may be optional.

The well 2 in an optional shape may be formed by scanning the well formation surface 1A of the substrate 5 of PDMS with the electron beam in a beam form.

Although a cell culturing dish in a plate shape is exemplarily described above as the cell culturing instrument 1, the shape and usage of a culturing instrument to which the present invention is applied are optional. For example, what is called a cell culturing plate and a microtiter plate are exemplary application targets.

The substrate for test use according to the present invention is not limited to the cell culturing instrument 1.

Specifically, the substrate for test use according to the present invention in which traps are fixed at the surface of the well 2 is widely applicable to usage for detection of a particular living body material, and is applicable to a bio chip, such as a DNA chip or a protein chip, in which traps such as probe DNA and antibody are fixed at the surface of the well 2.

In addition, the substrate for test use according to the present invention is also applicable as a substrate of a micro flow path chip or the like.

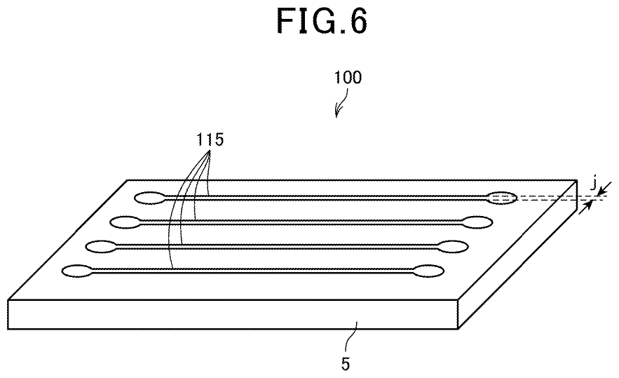

FIG. 6 is a diagram illustrating an exemplary micro flow path chip 100. In the micro flow path chip 100, a plurality of flow paths 115 are formed as solution retaining parts at the surface of the substrate 5 in place of the well 2. Each flow path preferably has a width j of 10 μm to 100 μm.

In the micro flow path chip 100 and the above-described bio chip, the values of the flow paths 115, the maximum depth d of the concave part of the well 2, the thickness f of the surface layer 6, and the like are same as those of the cell culturing instrument 1 described in the embodiment.

### REFERENCE SIGNS LIST

1 cell culturing instrument (substrate for test use)

1A well formation surface (surface)

2 well (solution retaining part)

4 culture media

6 surface layer

5 substrate

100 micro flow path chip (substrate for test use)

115 flow path (solution retaining part)

d maximum depth

f maximum thickness of surface layer

ϕ opening diameter

1. A substrate for test use in which a solution retaining part for retaining water or an aqueous solution is formed at a surface of the substrate, wherein
the substrate is made of polydimethylsiloxane (PDMS),
the solution retaining part is a concave part having a hydrophilic surface layer,
the surface layer of the concave part has a water contact angle that is smaller than a water contact angle of a non-concave part in the surface of the substrate and a thickness of the thickest part of the hydrophilic surface layer of the concave part is 20 μm or larger.

2. The substrate for test use according to claim 1, wherein the concave part has a maximum depth of 0.5 μm or larger.

3. The substrate for test use according to claim 1, wherein the solution retaining part has wettability with a water contact angle of 90° or smaller.

4. A method for manufacturing a substrate for test use, in which a solution retaining part that retains water or a solution is formed, the method comprising an electron beam irradiation process of irradiating a place which is of the substrate made of polydimethylsiloxane (PDMS) and at which the solution retaining part is to be formed with an electron beam at an acceleration voltage of the electron beam, which is equal to or lower than 1 MV and a radiation dose, of the electron beam, which is equal to or larger than 2 MGy, wherein
in the electron beam irradiation process, by irradiating the electron beam, a concave part that retains the water or the solution is formed and a surface layer reformed to be hydrophilic is formed at a surface of the concave part.

5-6. (canceled)

7. The method for manufacturing a substrate for test use according to claim 4, wherein, in the electron beam irradiation process, the surface of the substrate is irradiated with the electron beam in an atmosphere having an oxygen concentration equal to or higher than an oxygen concentration of air atmosphere.

8. The substrate for test use according to claim 1, wherein the water contact angle of the hydrophilic surface layer of the concave part is smaller than the water contact angle of the non-concave part in the surface of the substrate by 10 degrees or more.

9. The substrate for test use according to claim 1, wherein the water contact angle of the surface layer of the concave part that has been left for 10 days in an environment at 37° C. in a state that the concave part has retained the water or the aqueous solution, is within a range of the water contact angle±15% of the surface layer of the concave part before being left.

10. The substrate for test use according to claim 1, wherein the concave part has a maximum depth of 1 μm or larger and 50 μm or smaller and an opening with a size of 5 μm or larger and 100 μm or smaller.

11. The substrate for test use according to claim 1, wherein the substrate is provided in a bottom surface of a cell culturing instrument having a flat-bottomed dish shape, the solution retaining part is formed in the bottom surface.
